(19) United States
(12) Patent Application Publication
(10) Pub. No.: US 2008/0153408 A1
(43) Pub. Date: Jun. 26, 2008

(54) AIR REFRESH SYSTEM

(75) Inventor: Daniel Scott Thomas, Eastpointe, MI (US)

Correspondence Address: BROOKS KUSHMAN P.C., 1000 TOWN CENTER, TWENTY-SECOND FLOOR, SOUTHFIELD, MI, 48075, US

(73) Assignee: LIVERNOIS VEHICLE DEVELOPMENT, LLC, Inkster, MI (US)

(21) Appl. No.: 11/614,319

(22) Filed: Dec. 21, 2006

(51) Int. Cl. B60H 3/00; A61L 9/00

(52) U.S. Cl. 454/156; 422/123; 422/105; 422/5

(57) ABSTRACT

An air refresh system (10) for use in a occupant compartment. The system (10) includes a pressurized container assembly (12) for storing a pressurized refresh fluid (14), one or more nozzles (20) for dispersing the pressurized refresh fluid (14) and one or more electronically activated valves (22) for controlling the flow of the pressurized refresh fluid (14) from the container assembly (12) to the nozzles (20). The system (10) also has a microprocessor-based controller (24) programed to: receive one ore more user input signals (28), process the one or more user input signals (28) to obtain one or more fluid control signals (34), and transmit the one or more fluid control signals (34) to the one or more electronically activated valves (22) to allow the pressurized refresh fluid (14) to flow from the container assembly (12) to the one or more nozzles (20), wherein the one or more nozzles (20) spray the pressurized refresh fluid (14) into the vehicle to refresh air in the vehicle.

### BACKGROUND OF THE INVENTION

1. Field of the Invention

The invention relates to an refresh system and method for refreshing the interior of a vehicle.

2. Background Art

U.S. Pat. No. 5,078,046 discloses an air treatment apparatus and method for a vehicle. The apparatus has a pump timer arranged so as to afford a finite delay, such as 10 seconds, to enable a fan motor to be actuated and a fan to be operating at a speed suitable to create a desired air flow in a duct interior before discharge is attempted. After this predetermined delay, the pump timer energizes a pump to deliver one or more “shots” of liquid from a container to a spray nozzle, where it is discharged in an atomized condition. In the example given, a single continuous spray of five seconds would be appropriate. Thereafter, the pump shot timer clocks out and the pump motor relay is de-energized; the fan timer, according to this example, would call for continued operation of the fan motor for additional 30 seconds, i.e., until the entire operating duration had timed out. Thereupon, the fan motor relay would drop out and the fan would be de-energized.

Other prior art patents include U.S. Pat. Nos. 7,013,656; 6,494,778; 6,264,548; 6,048,265; 5,958,346; 5,882,256; 5,833,929; 5,297,988; 5,171,485; 5,078,046; 4,913,034; 4,867,045; 4,805,520; 4,677,902; 4,352,321; 4,309,382; 3,891,149; and 3,259,050.

Applicant's invention improves on the above designs as summarized below.

### SUMMARY OF THE INVENTION

One aspect of the present invention is to provide a more effective, cost efficient and environmentally conscious air refresh system. The term refresh refers to dispersing a pressurized refresh fluid (i.e., either a liquid or gas) into an interior of a vehicle. The fluid may be use to either sanitize, refresh, or both sanitize and refresh the interior of the vehicle. For example, the fluid may be an disinfectant, antibacterial agent, fungicide, anti-mildew agent, or deodorizing agent.

The air refresh system of the vehicle includes a pressurized container assembly for storing the pressurized refresh fluid. The air refresh system has one or more nozzles for dispersing the pressurized refresh fluid into the vehicle to refresh air in the vehicle. In addition, the air refresh system includes one or more electronically activated valves for controlling the flow of the pressurized refresh fluid from the container assembly to the nozzles and a microprocessor-based controller. The microprocessor-based controller is programed to receive one or more user input signals and to process the user input signals to obtain one or more fluid control signals. Furthermore, the microprocessor-based controller is programmed to transmit the fluid control signals to the electronically activated valves to allow the pressurized refresh fluid to flow from the container assembly to the nozzles.

Other advantages and useful embodiments are disclosed or suggested in the claims, the description of the figures, and the drawings.

### BRIEF DESCRIPTION OF THE DRAWINGS

FIG. 1 is a schematic diagram illustrating an air refresh system having a pressurized container assembly, a plurality of electronically activated valves, a plurality of nozzles, and a microprocessor-based controller; and

FIG. 2 is a flowchart diagram illustrating a method of refreshing air located within an occupant compartment.

### DETAILED DESCRIPTION OF THE PREFERRED EMBODIMENT(S)

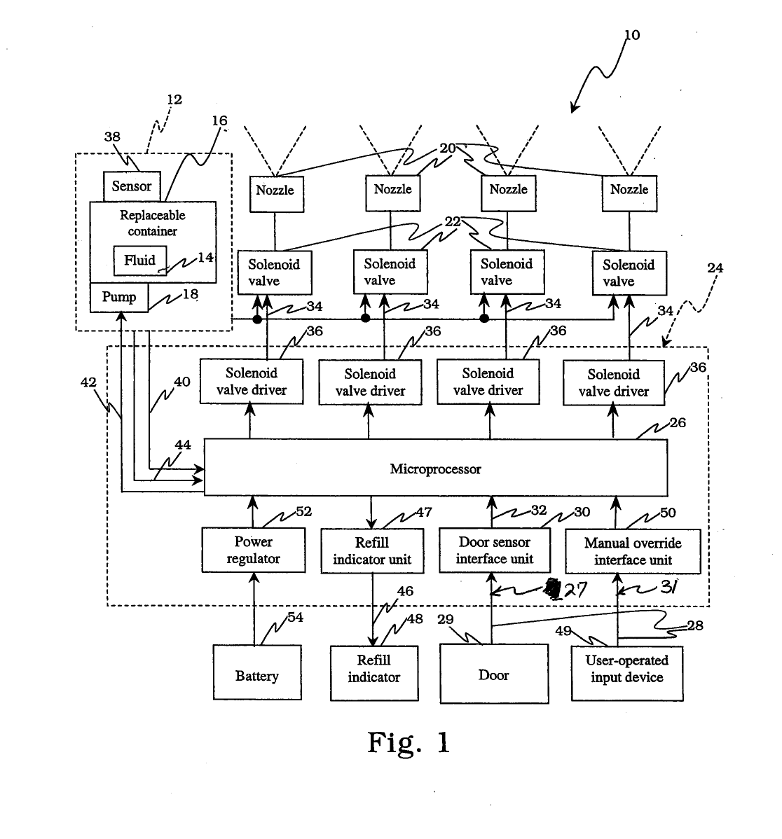

With reference to FIG. 1, an air refresh system 10 of a vehicle in accordance with the present invention includes a pressurized container assembly 12 for storing a pressurized refresh fluid 14. In the preferred construction of the air refresh system 10, the container assembly 12 includes a replaceable container 16 for storing the pressurized refresh fluid 14. The pressurized refresh fluid 14 may be a sanitizer fluid. The sanitizer fluid may be a disinfectant, an antibacterial agent, a fungicide, an anti-mildew agent, a deodorizing agent, or a combination of two or more of these ingredients. Furthermore, the container assembly 12 may include a pump 18 for pressurizing the fluid 14 within the container assembly 12. The pump 18 may also be used to pressurize the fluid 14 within the replaceable container 16 of the container assembly 12.

With continuing reference to FIG. 1, the air refresh system 10 includes one or more nozzles, which is preferably a plurality of nozzles 20, for dispersing the pressurized refresh fluid 14. The plurality of nozzles 20 may be used to spray the refresh fluid 14 into one or more ducts (not shown) of a heat ventilation air conditioning (HVAC) system (not shown) of the vehicle. The ducts of the HVAC system may be used to transport the refresh fluid 14 from the plurality of nozzles 20 to an occupant compartment area (not shown) of the vehicle. Furthermore, the refresh fluid 14 may be sprayed into the ducts of the HVAC system while a blower unit (not shown) of the HVAC system moves air through the ducts of the HVAC system. The blower unit may move air through the ducts to facilitate transportation of the refresh fluid 14 from the plurality of nozzles 20 to the occupant compartment area of the vehicle to refresh air in the vehicle. In addition, the blower unit may continue to move air through the one or more ducts of the HVAC system after the plurality of nozzles 20 stop dispersing the pressurized refresh fluid 14. The blower unit may continue to move air through the ducts in order to move more of the pressurized refresh fluid 14 out of the ducts to provide a more complete air refresh of the occupant compartment area. For example, the blower may start blowing air through the ducts of the HVAC system for one and one-half seconds before the nozzles 20 spray the pressurized refresh fluid 14 for an one-half second and stop blowing air through the ducts for four and one-half seconds after the nozzles 20 stop dispersing the pressurized refresh fluid 14.

As illustrated in FIG. 1, the air refresh system 10 includes one or more electronically activated valves, which are preferably a plurality of electronically activated valves 22, for controlling the flow of the pressurized refresh fluid 14 from the container assembly 12 to the plurality of nozzles 20. The plurality of electronically activated valves 22 may include one or more digital electronically activated valves such as an X-Valve® of Parker Hannifin Corporation.

With continuing reference to FIG. 1, the air refresh system 10 has a microprocessor-based controller 24. The microprocessor-based controller 24 includes a microprocessor 26. The controller 24 is programed to receive one or more user input signals 28. For example, the user input signals 28 may include one or more door input signals 27, which are generated when a door 29 of the vehicle is opened. Alternatively, the user input signals 28 may include a manual signal 31, which is generated when an ignition of a vehicle (not shown) is turned on. Furthermore, the controller 24 may include a door sensor interface unit 30 to receive and process each of the door input signals 27 to obtain a processed signal 32. In addition, the microprocessor-based controller 24 is programed to process the user input signals 28 to obtain the one or more fluid control signals 34. For example, the controller 24 may process the door input signals 27 to obtain fluid control signals 34. The microprocessor-based controller 24 is also programed to transmit the one or more fluid control signals 34 to the plurality of electronically activated valves 22. The fluid control signals 34 are transmitted to the electronically activated valves 22 to allow the pressurized refresh fluid 14 to flow from the container assembly 12 to the plurality of nozzles 20. The controller 24 may transmit the fluid control signals 34 to each of the plurality of electronically activated valves 22 so that each of the plurality of nozzles 20 sprays the refresh fluid 14 at different time intervals. In addition, the controller 24 of the air refresh system 10 may have one or more electronically activated valve drivers 36. The electronically activated valve drivers 36 of the controller 24 may be use to transmit the fluid control signals 34 from the controller 24 to the plurality of electronically activated valves 22.

With continuing reference to FIG. 1, the pressurized container assembly 12 may include a sensor 38 for monitoring the pressurized refresh fluid 14. The sensor 38 monitors the pressure of the refresh fluid 14 and/or the amount of the refresh fluid 14 fluid in the replaceable container 16. If the pressure of the refresh fluid 14 is below a predetermined pressure threshold, then the container assembly 12 transmits a low pressure signal 40 from the container assembly 12 to the microprocessor-based controller 24. The microprocessor-based controller 24 receives the low pressure signal 40 and transmits a pump initiation signal 42 to the pump 18 of the container assembly 12. The pump 18 receives the pump initiation signal 42 and activates to pressurize the fluid 14 within the container assembly 12 above the predetermined pressure threshold. In addition, the sensor 38 of the pressurized container assembly 12 may be used to send an low fluid signal 44 to the microprocessor-based controller 24 when the refresh fluid 14 in the container assembly 12 is below a refill threshold amount.

With continuing reference to FIG. 1, the microprocessor-based controller 24 may be programmed to delay transmitting the fluid control signals 34 to the electronically activated valves 22 for a delay time interval. The fluid control signals 34 may be delayed to the electronically activated valves 22 to prevent the nozzles 20 from dispersing the fluid 14 during the delay time interval. The microprocessor-based controller 24 calculates the delay time interval from a time when the controller 24 last transmitted the one or more fluid control signals 34 to a time when the controller 24 begins to calculate the delay time interval. For example, when the door 29 of the vehicle is opened, the controller 24 may receive and process the one or more door input signals 27 and transmit one or more fluid control signals 34 based on the door input signals 27. Similarly, when the ignition of a vehicle is turned on, the controller 24 may receive and process the manual signal 31 and transmit one or more fluid control signals 34. When the controller 24 transmits the fluid control signals 34, the controller 24 starts to calculate the delay time interval until the controller 24 transmits subsequent fluid control signals 34. In addition, the air refresh system 10 may have the controller 24 programmed to transmit a refill signal 46 to a refill indicator 48 when the controller 24 either counts a predetermined number of sprays or when the refresh fluid 14 in the container assembly 12 of the container assembly 12 is below a refill threshold amount.

As illustrated in FIG. 1, the air refresh system 10 may have an user-operated input device 49 for providing the manual signal 31 to a manual override interface unit 50 of the controller 24. A user of the air refresh system 10 may choose to operate the user-operated input device 49 whenever the user wants to refreshen to the occupant compartment area of the vehicle independent of the controller 24 receiving the door input signals 27. In addition, the air refresh system 10 may have the controller 24 programmed to transmit the refill signal 46 to the refill indicator 48 when the controller 24 either counts a predetermined number of sprays or when the refresh fluid 14 in the container assembly 12 or replaceable container 16 of the container assembly 12 is below a refill threshold amount. The refill indicator 48 may be light emitting diode (LED) or some other light emitting device. Furthermore, the controller 24 may include a refill indicator unit 47 for transmitting the refill signal 46 from the controller 24 to the refill indicator 48. In addition, the controller 24 of the air refresh system 10 may have a power regulator 52 that regulates electrical power from a battery 54 of the vehicle to provide regulated electrical power for the controller 24.

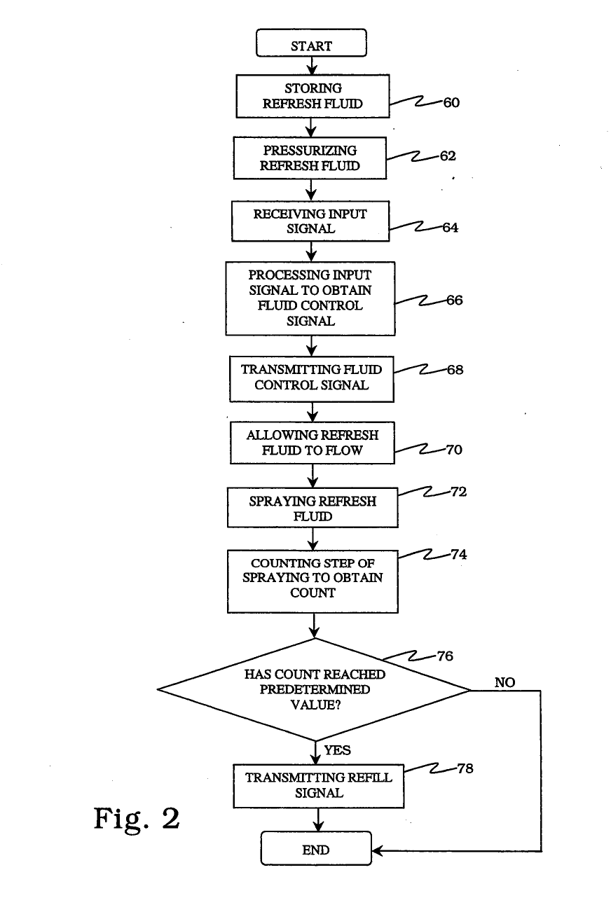

With reference to FIG. 2, a method of refreshing air of an occupant compartment is illustrated. In block 60, a pressurized refresh fluid 14 is stored in a pressurized container assembly 12. For example the pressurized refresh fluid 14 may be stored in a replaceable container 16 of the pressurized container assembly 12. In block 62, a pump 18 pressurizes the pressurized refresh fluid 14 within the pressurized container assembly 12 prior to receiving one or more user input signals 28. In block 64, a microprocessor-based controller 24 receives the user input signals 28. The user input signals 28 may be generated from an user-operated input device 49. Alternatively, the user input signals 28 may be generated when a door 29 of the vehicle is opened. In block 66, the microprocessor-based controller 24 processes the user input signals 28 to obtain one or more fluid control signals 34. In block 68, the fluid control signals 34 are transmitted from the controller 24 to the electronically activated valves 22. Furthermore, one or more electronically activated valve drivers 36 of the controller 24 may transmit the fluid control signals 34 from the controller 24 to the electronically activated valves 22. In block 70, the one or more electronically activated valves, which are preferably a plurality of electronically activated valves 22, allow the pressurized refresh fluid 14 to flow from the pressurized container assembly 12 to one or more nozzles, which are preferably a plurality of nozzles 20, based on the transmitted fluid control signals 34. In block 72, the plurality of nozzles 20 spray the flowing pressurized refresh fluid 14 into the occupant compartment to refresh air in the vehicle. In block 74, the microprocessor-based controller 24 counts the step of dispersing to obtain a count. At decision 76, the microprocessor-based controller 24 decides whether the count has reached a predetermined value. The predetermined value represents the number of sprays allowed before the pressurized refresh fluid 14 should be replaced within a replacement time interval. If the count has reached the predetermined value, then the controller 24 transmits a refill signal 46 to a refill indicator 48. For example, a refill indicator unit 47 of the controller 24 may transmit the refill signal 46 to the refill indicator 48 to alert a user of the air refresh system 10 that the refresh fluid 14 should be replaced within a replacement time interval.

While embodiments of the invention have been illustrated and described, it is not intended that these embodiments illustrate and describe all possible forms of the invention. Rather, the words used in the specification are words of description rather than limitation, and it is understood that various changes may be made without departing from the spirit and scope of the invention.

1. An air refresh system for use in an occupant compartment, the system comprising:
a pressurized container assembly for storing a pressurized refresh fluid;
at least one nozzle for dispersing the fluid;
at least one electronically activated valve for controlling the flow of the pressurized refresh fluid from the container assembly to the at least one nozzle; and
a microprocessor-based controller programed to: receive at least one user input signal, process the at least one user input signal to obtain at least one fluid control signal, and transmit the at least one fluid control signal to the at least one electronically activated valve to allow the pressurized refresh fluid to flow from the container assembly to the at least one nozzle, wherein the at least one nozzle sprays the pressurized refresh fluid into the vehicle to refresh air in the vehicle.

2. The air refresh system as in claim 1, wherein the microprocessor-based controller includes a door sensor interface unit for processing the at least one user input signal to obtain a signal, wherein the at least one user input signal is at least one door input signal for determining when a door of the vehicle is opened.

3. The air refresh system as in claim 2, wherein the controller is programmed to receive a plurality of door input signals and wherein the door sensor interface unit of the controller receives each of the plurality of door input signals.

4. The air refresh system as in claim 1, wherein the controller is programmed to delay transmitting the at least one fluid control signal to the at least one electronically activated valve for a delay time interval to prevent the at least one nozzle from dispersing the fluid during the delay time interval, wherein the delay time interval is calculated by the controller from a time when the controller last transmitted the at least one fluid control signal to a time when the controller begins to calculate the delay time interval.

5. The air refresh system as in claim 1, wherein an user-operated input device provides the at least one user input signal to a manual override interface unit of the controller, wherein the controller is programmed to receive and process the at least one user input signal and to transmit the at least one fluid control signal to the at least one electronically activated valve.

6. The air refresh system as in claim 1, wherein the controller is programmed to transmit a refill signal to a refill indicator when the controller counts a predetermined number of sprays.

7. The air refresh system as in claim 1, wherein the container assembly includes a replaceable container for storing the fluid and the controller transmits a refill signal to a refill indicator when the fluid in the replaceable container is below a refill threshold amount.

8. The air refresh system as in claim 1, wherein the container assembly includes a replaceable container for storing the fluid and a pump for pressurizing the fluid within the container.

9. The air refresh system as in claim 1, wherein the system includes a plurality of electronically activated valves and a plurality of nozzles, wherein the controller transmits a plurality of fluid control signals to each of the plurality of electronically activated valves so that each of the plurality of nozzles sprays the fluid at different time intervals.

10. The air refresh system as in claim 1, wherein the pressurized refresh fluid is a sanitizer fluid.

11. The air refresh system as in claim 10, wherein the sanitizer fluid is selected from the group consisting of a disinfectant, an antibacterial agent, a fungicide, an anti-mildew agent, and a deodorizing agent.

12. The air refresh system as in claim 1, wherein the at least one nozzle sprays the fluid in at least one duct of a heat ventilation air conditioning (HVAC) system of the vehicle while a blower unit of the HVAC system moves air through the at least one duct of the HVAC system.

13. The air refresh system as in claim 12, wherein the blower unit continues to move air through the at least one duct of the HVAC system after the at least one nozzle stops dispersing the fluid.

14. The air refresh system as in claim 13, wherein the blower unit of the HVAC system starts to move air through the at least one duct about one second before the at least one nozzle starts to spray the fluid, and stops moving air through the at least one duct until about four and a half seconds after the at least one nozzle stops dispersing the fluid.

15. The air refresh system as in claim 1, wherein a power regulator of the controller regulates electrical power from a battery of the vehicle to provide regulated electrical power for the controller.

16. An air refresh system for use in an occupant compartment, the system comprising:
a pressurized container assembly for storing a pressurized refresh fluid;
at least one nozzle for dispersing the fluid into at least one duct of a heat ventilation air conditioning (HVAC) system of the vehicle while a blower unit of the HVAC system moves air through the at least one duct of the HVAC system to refresh air in the vehicle, wherein the blower unit continues to move air through the at least one duct of the HVAC system after the at least one nozzle stops dispersing the pressurized refresh fluid;
at least one electronically activated valve for controlling the flow of the pressurized refresh fluid from the container assembly to the at least one nozzle;
a microprocessor-based controller programed to: receive at least one user input signal, process the at least one user input signal to obtain at least one fluid control signal, and transmit the at least one fluid control signal to the at least one electronically activated valve to allow the pressurized refresh fluid to flow from the container assembly to the at least one nozzle, wherein the microprocessor-based controller includes a door sensor interface unit for processing the at least one user input signal to obtain a signal, wherein the at least one user input signal is at least one door input signal for determining when a door of the vehicle is opened; and
an input device for providing the at least one user input signal to a manual override interface unit of the controller, wherein the controller is programmed to receive and process the at least one user input signal and to transmit the at least one fluid control signal to the at least one electronically activated valve.

17. A method of refreshing air of an occupant compartment, the method comprising:
storing and pressurizing a refresh fluid;
receiving at least one user input signal;
processing the at least one user input signal to obtain at least one fluid control signal;
transmitting the at least one fluid control signal;
allowing the stored pressurized refresh fluid to flow based on the at least one transmitted fluid control signal; and
dispersing the flowing pressurized refresh fluid into the vehicle to refresh air in the vehicle.

18. The method as in claim 18 wherein the at least one user input signal is generated when a door of the vehicle is opened.

19. The method as in claim 17 further comprising:
counting how often the step of dispersing is performed to obtain a count; and
transmitting a refill signal when the count reaches a predetermined value.
